(19) United States
(12) Patent Application Publication
(10) Pub. No.: US 2019/0112451 A1
(43) Pub. Date: Apr. 18, 2019

(54) RADIATION-RESISTANT RESIN ADDITIVE, RADIATION-RESISTANT MEDICAL POLYAMIDE RESIN COMPOSITION, AND RADIATION-RESISTANT MEDICAL MOLDED ARTICLE

(71) Applicant: KANEKA CORPORATION, Osaka-shi, Osaka (JP)

(72) Inventor: Takayuki KATO, Settsu-shi (JP)

(73) Assignee: KANEKA CORPORATION, Osaka-shi, Osaka (JP)

(21) Appl. No.: 16/091,490

(22) PCT Filed: Mar. 10, 2017
(86) PCT No.: PCT/JP2017/009823

§ 371 Date: Oct. 4, 2018

(30) Foreign Application Priority Data
Apr. 6, 2016 (JP) 2016-076807

(51) Int. Cl. C08K 5/13; C08G 69/40; C08G 69/36; C08L 77/00; A61L 29/04

(52) U.S. Cl. CPC C08K 5/13; C08G 69/40; C08G 69/36; C08L 2203/02; A61L 29/04; A61L 2400/00; C08L 2201/08; C08L 77/00

(57) ABSTRACT

A radiation-resistant resin additive that contains, as an effective ingredient, a bisphenol compound as indicated by General Formula (1) below, or General Formula (2) below, as well as a radiation-resistant medical polyamide resin composition comprising the foregoing bisphenol compound and an amide resin, and a radiation-resistant medical molded article fabricated using the foregoing radiation-resistant medical polyamide resin composition.

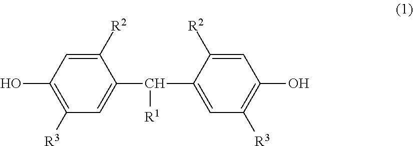

(In the Formula, R¹, R², and R³ respectively may be the same or different and indicate a hydrogen atom or a saturated hydrocarbon group having not less than 1 carbon.)

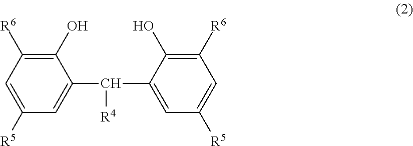

(In the Formula, R⁴, R⁵, and R⁶ respectively may be the same or different and indicate a hydrogen atom or a saturated hydrocarbon group having not less than 1 carbon.)

### TECHNICAL FIELD

The present invention relates to a radiation-resistant resin additive, a radiation-resistant medical polyamide resin composition, and a radiation-resistant medical molded article.

### BACKGROUND ART

In the medical field, a variety of medical materials are used which are polymer materials. Medical equipment manufactured from such materials must ultimately be sterilized. As such sterilization method, autoclave sterilization, ethylene oxide gas (EOG) sterilization, and radiation sterilization are primarily employed.

Of these, autoclave sterilization is not very widely employed. If the medical equipment is processed under high temperature and high pressure, such as in the autoclave sterilization, the polymer material would deteriorate or alter. As EOG sterilization causes little deterioration of the polymer material, it had until recently been widely employed. However, it has in recent years been indicated that EOG remains in the medical material following EOG sterilization, and that this has an adverse effect on living organisms. Accordingly, radiation sterilization has recently become the subject of attention.

Radiation sterilization is a method for sterilizing medical equipment by applying, thereto, gamma rays and/or electron beams. Indeed, it is possible to kill microorganisms and so forth that remain in medical equipment by applying radiation. However, it has been indicated that formation of crosslinks between macromolecule chains occurs in the polymer material contained within component parts of medical equipment, or conversely, cleavage of the molecular chains therein. This causes alteration of properties such as a decrease in macromolecule strength and/or change in percent elongation, and adversely effects on medical equipment performance.

To address the above, Patent Reference 1 discloses a medical material comprising a composition in which a polyfunctional triazine compound is made to be present within a resin component.

### PRIOR ART REFERENCES

### Patent References

Patent Reference 1: Japanese Patent Application Publication No. 2003-695

### SUMMARY OF INVENTION

### Problem to be Solved by Invention

However, even where resin compositions containing polyfunctional triazine compounds such as are disclosed in Patent Reference No. 1 have been employed, it has been indicated that radiation resistance is inadequate especially with respect to high-intensity electron beams, and there has been a demand for improvement thereof. It is therefore an object of the present invention to provide a radiation-resistant resin additive, a radiation-resistant medical polyamide resin composition, and a radiation-resistant medical molded article, which have superior radiation resistance with respect to high-intensity radiation, without decreasing strength/elongation relative to that originally possessed by the resin.

### Means for Solving Problem

The present inventor(s) engaged in intensive study for the purpose of solving the aforementioned problems. As a result of the study, the following was discovered. Autoagglutination of the polyfunctional triazine compound disclosed in Patent Reference No. 1 is intense and the melting point thereof is 80° C. or less. When such a polyfunctional triazine compound is added to, for example, aliphatic polyamide resin, or to polyolefin or other such resin or the like, which are substantially different from of the polyfunctional triazine compound in melting point and melt viscosity, the substantial difference causes homogeneous dispersion within the resin matrix to be difficult. In addition, if this is not homogeneously dispersed therewithin, it was thought that (1) were irradiation with radiation carried out, reaction for radical crosslinking between polyfunctional triazine compound sites would hardly proceed at all, but self-reaction would proceed in abundant fashion, and as this would represent contamination to the resin, there would be a decrease in strength/elongation, and (2) were irradiation with radiation not carried out, there would be a possibility that the inhomogeneous dispersion would also cause decrease in strength/elongation. Upon further study, it was discovered that the aforementioned problems could be solved through employment of certain bisphenol compound(s), which led to the perfection of the present invention. That is, the present invention relates to the radiation-resistant medical polyamide resin composition at [1] through [9] below; the radiation-resistant medical molded article at [10] and [11]; and the radiation-resistant resin additive at [12].

[1] A radiation-resistant medical polyamide resin composition comprising: (a) a bisphenol compound indicated by General Formula (1) below, or General Formula (2) below; and (b) an amide resin.

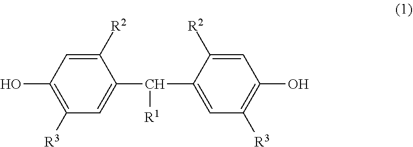

(In the Formula, R¹, R², and R³ respectively may be the same or different and indicate a hydrogen atom or a saturated hydrocarbon group having not less than 1 carbon.)

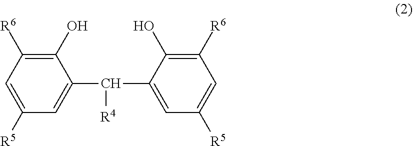

(In the Formula, R⁴, R⁵, and R⁶ respectively may be the same or different and indicate a hydrogen atom or a saturated hydrocarbon group having not less than 1 carbon.)

[2] The radiation-resistant medical polyamide resin composition according to the foregoing [1] wherein the (a) bisphenol compound is present therein in an amount that is 0.01 wt % to 10 wt %.

[3] The radiation-resistant medical polyamide resin composition according to the foregoing [1] or [2] wherein the (a) bisphenol compound is 4,4′-butylidenebis-(6-t-butyl-3-methylphenol) or 2,2′-methylenebis-(4-ethyl-6-t-butylphenol).

[4] The radiation-resistant medical polyamide resin composition according to any one of the foregoing [1] through [3] wherein the (b) amide resin has a structure derived from at least one species selected from among polyoxyalkylene glycol and (b1) polyether diamine as soft segment, and has a structure derived from at least one species of (b2) carboxylic-acid-terminated polyamide as hard segment.

[5] The radiation-resistant medical polyamide resin composition according to the foregoing [4] wherein the (b2) carboxylic-acid-terminated polyamide has a structure derived from at least one species of (b21) aminocarboxylic acid indicated by General Formula (3), below, and has a structure derived from at least one species of (b22) dicarboxylic acid indicated by General Formula (4), below.

HOOC—R⁷NH—CO—R⁷ₙNH₂ (3)

(In the Formula, R⁷ indicates saturated hydrocarbon group(s), each of which respectively has not less than 1 carbon, and n indicates an integer not less than 0. Furthermore, when there are two or more species of repeating units that contain R⁷, n is the sum of all of the respective repeating units that contain R⁷.)

HOOC—R⁸—COOH (4)

(In the Formula, R⁸ indicates a direct bond or a saturated hydrocarbon group having not less than 1 carbon.)

[6] The radiation-resistant medical polyamide resin composition according to the foregoing [4] or [5] wherein the (b1) polyether diamine is at least one species indicated by General Formula (5) below.

H₂NR⁹—OₘR¹⁰—NH₂ (5)

(In the Formula, R⁹ independently indicates saturated hydrocarbon group(s), each of which has not less than 1 carbon, R¹⁰ indicates a saturated hydrocarbon group having not less than 1 carbon and m indicates an integer not less than 1. Furthermore, when there are two or more species of repeating units that contain R⁹, m is the sum of all of the respective repeating units that contain R⁹.)

[7] The radiation-resistant medical polyamide resin composition according to any one of the foregoing [4] through [6] wherein the (b1) polyether diamine is at least one species indicated by General Formula (6) below.

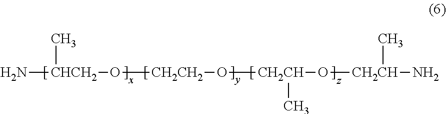

(In the Formula, x+z indicates an integer not less than 1, and y indicates an integer not less than 1.)

[8] The radiation-resistant medical polyamide resin composition according to any one of the foregoing [4] through [7] wherein the (b) amide resin has: a structure derived from at least one species selected from among the polyoxyalkylene glycol and the (b1) polyether diamine; a structure derived from at least one species of the (b2) carboxylic-acid-terminated polyamide; and a structure derived from at least one species of (b3) diamine indicated by General Formula (7) below.

H₂N—R¹¹—NH₂ (7)

(In the Formula, R¹¹ indicates the saturated hydrocarbon group having not less than 1 carbon.)

[9] The radiation-resistant medical polyamide resin composition according to any one of the foregoing [4] through [8] wherein the number average molecular weight (Mn) of the (b2) carboxylic-acid-terminated polyamide is not less than 4000.

[10] A radiation-resistant medical molded article fabricated using a resin composition containing the radiation-resistant medical polyamide resin composition according to any one of the foregoing [1] through [9].

[11] A radiation-resistant medical molded article according to the foregoing [10], which is a medical tube or a medical balloon.

[12] A radiation-resistant resin additive that contains, as an effective ingredient, a bisphenol compound as indicated by General Formula (1), below, or General Formula (2) below.

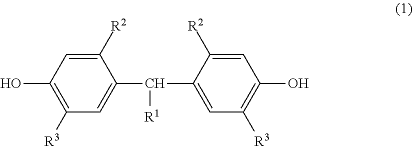

(In the Formula, R¹, R², and R³ respectively may be the same or different and indicate a hydrogen atom or a saturated hydrocarbon group having not less than 1 carbon.)

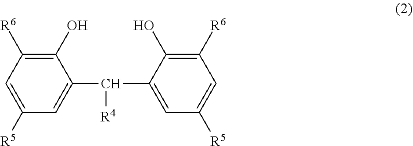

(In the Formula, R⁴, R⁵, and R⁶ respectively may be the same or different and indicate a hydrogen atom or a saturated hydrocarbon group having not less than 1 carbon.)

### BENEFIT OF THE INVENTION

The present invention makes it possible to provide a radiation-resistant resin additive, a radiation-resistant medical polyamide resin composition, and a radiation-resistant medical molded article, which have superior radiation resistance with respect to high-intensity radiation, without decreasing strength/elongation relative to that originally possessed by the resin.

### EMBODIMENTS FOR CARRYING OUT THE INVENTION

Although description is given below in terms of radiation-resistant resin additives, radiation-resistant medical polyamide resin compositions, and radiation-resistant medical molded articles that are associated with embodiments of the present invention, the present invention is not to be limited thereby.

Radiation-Resistant Resin Additive

The foregoing radiation-resistant resin additive contains bisphenol compound(s) indicated by General Formula (1) and/or General Formula (2) below, as effective ingredient(s).

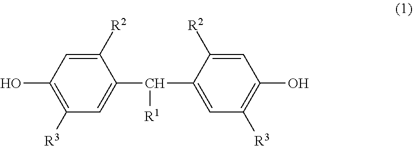

(In the Formula, R¹, R², and R³respectively may be the same or different and indicate a hydrogen atom or a saturated hydrocarbon group having not less than 1 carbon.)

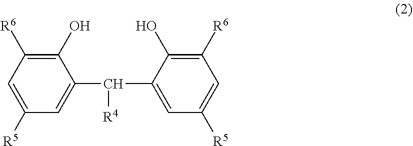

(In the Formula, R⁴, R⁵, and R⁶ respectively may be the same or different and indicate a hydrogen atom or a saturated hydrocarbon group having not less than 1 carbon.)

A resin additive containing bisphenol compound(s) having such structure(s) as effective ingredient(s) is such that addition thereof to any of various resin compositions will make it possible to effectively suppress alteration of properties of such resin composition(s) despite irradiation thereof with gamma rays, electron beams, and/or other such radiation. That is, there will be no decrease in strength/elongation relative to that originally possessed by the resin. It is thought that such function prevents crosslinks between molecular chains and/or cleavage of molecular chains in polymer compound(s) contained within the resin composition by capturing radicals generated at the time when the resin composition is irradiated with radiation, or by preventing the generation of radicals, as a consequence of satisfactory dispersion characteristics of bisphenol compound(s) with respect to various resins.

As bisphenol compounds indicated by General Formula (1), it is sufficient that R¹, R², and R³ in Formula (1) be a hydrogen atom or a saturated hydrocarbon group having not less than 1 carbon. The saturated hydrocarbon group may be of either a chain-like or ring-like structure. From the standpoint of chemical interactions with the resin composition, a chain-like structure is preferred. Where a chain-like structure is employed, this may be a straight-chain or it may be branched. Furthermore, as the saturated hydrocarbon group having not less than 1 carbon, from the standpoint of radiation resistance it is preferred that the number of carbons be not less than 1 but not greater than 8, and more preferred that this be not less than 1 but not greater than 6. As a combination of such saturated hydrocarbon groups, it is preferred that R¹ be hydrogen atom or be such that the number of carbons is not less than 1 but not greater than 4, it is preferred that R² be a hydrogen atom or be such that the number of carbons is not less than 1 but not greater than 4, and it is preferred that R³ be hydrogen atom or be such that the number of carbons is not less than 1 but not greater than 4. Thereamong, it is particularly preferred that R¹ be a propyl group for which the number of carbons is 3, that R² be a methyl group for which the number of carbons is 1, and that R³ be a butyl group for which the number of carbons is 4; as an example of which it is possible to cite 4,4′-butylidenebis-(6-t-butyl-3-methylphenol).

As bisphenol compounds indicated by General Formula (2), it is sufficient that R⁴, R⁵, and R⁶ at Formula (2) be a hydrogen atom or a saturated hydrocarbon group having not less than 1 carbon. The saturated hydrocarbon group may be of either a chain-like or ring-like structure. From the standpoint of chemical interactions with the resin composition, a chain-like structure is preferred. Where a chain-like structure is employed, this may be a straight-chain or it may be branched. As the saturated hydrocarbon group having not less than 1 carbon, from the standpoint of radiation resistance it is preferred that the number of carbons be not less than 1 but not greater than 8, and more preferred that this be not less than 1 but not greater than 4. As more preferred bisphenol compounds indicated by General Formula (2), R⁴ might be hydrogen atom, R⁵ might be hydrogen atom or a saturated hydrocarbon group at which the number of carbons is not less than 1 but not greater than 4, and R⁶ might be hydrogen atom or a saturated hydrocarbon group at which the number of carbons is not less than 1 but not greater than 6. Thereamong, it is particularly preferred that R⁴ be hydrogen atom, that R⁵ be an ethyl group for which the number of carbons is 2, and that R⁶ be a butyl group for which the number of carbons is 4; as an example of which it is possible to cite 2,2′-methylenebis-(4-ethyl-6-t-butylphenol).

As the aforementioned bisphenol compound, commercially available products may be used. Yoshinox BB, 425 manufactured by Mitsubishi Chemical Corporation and so forth may be cited as examples. Note that while these commercially available products may be used as radiation-resistant resin additives, they were not until this time known to be radiation-resistant.

The foregoing radiation-resistant resin additives may be used together with any of the various resins. Resins, there being no particular limitation with respect thereto, such as polyolefin resins, polyvinyl chloride resins, ABS resins, polyester resins, fluorocarbon resins, polyamide resins, polyimide resins, polyamide-imide resins, polyurethane resins, silicone resins, and so forth may be cited as examples. Thereamong, polyamide resins are suitable. Furthermore, among polyamide resins, polyamide elastomers are more suitable.

Any of various additives may be added as necessary to the foregoing radiation-resistant resin additives.

The foregoing radiation-resistant resin additives may be employed in constituent material(s) for molded article(s) distributed after being subject to a step of irradiation with radiation. There being no particular limitation with respect to the types of radiation that may be used for irradiation, ions, electrons, protons, neutrons, and other such particle radiation, and gamma rays, x-rays, and other such electromagnetic radiation may be cited as examples. Thereamong, the foregoing radiation-resistant resin additive(s) are suitably employed in constituent material(s) for medical and/or other uses where sterilization processing is carried out by means of gamma rays, electron beams, and/or the like.

The amount(s) of radiation-resistant resin additive(s) which are added to resin(s) may be chosen as appropriate depending on the type(s) of radiation employed, conditions under which irradiation thereby takes place, resin composition, and so forth.

Radiation-Resistant Medical Polyamide Resin Composition

The foregoing radiation-resistant medical polyamide resin composition contains (a) bisphenol compound(s) indicated by General Formula (1) below, and/or General Formula (2) below; and (b) amide resin(s).

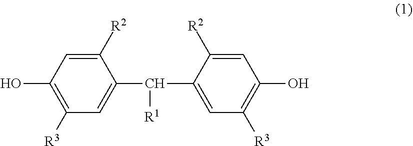

(In the Formula, R¹, R², and R³ respectively may be the same or different and indicate a hydrogen atom or a saturated hydrocarbon group having not less than 1 carbon.)

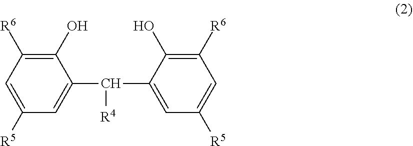

(In the Formula, R⁴, R⁵, and R⁶ respectively may be the same or different and indicate a hydrogen atom or a saturated hydrocarbon group having not less than 1 carbon.)

It is thought that the foregoing radiation-resistant medical polyamide resin composition, because it contains bisphenol compound(s) having such structure dispersed therewithin in a satisfactory state, permits interference with generation of radicals or capture of radicals generated when molded article(s) fabricated using the polyamide resin composition are irradiated with radiation, even where sterilization processing in the form of irradiation by gamma rays, electron beams, and/or other such radiation is carried out. This is thought to make it possible to prevent crosslinks between molecular chains and/or cleavage of molecular chains in polymer compound(s) contained within the polyamide resin composition that makes up the molded article(s), and effectively suppress alteration of properties of molded article(s) fabricated from the polyamide resin composition, which is to say that there will be no decrease in strength/elongation relative to that originally possessed by amide resin(s) and so forth included within the polyamide resin composition. This is likewise also true with respect to any radicals that remain following irradiation with radiation. Below, note that “radiation-resistant medical polyamide resin compositions” are sometimes referred to simply as “resin compositions”. Furthermore, “(a) bisphenol compounds indicated by General Formula (1) above, and/or General Formula (2) above, contained within radiation-resistant medical polyamide resin compositions” are sometimes referred to simply as “(a) bisphenol compounds”.

The (a) bisphenol compounds capable of being used in the context of the foregoing resin compositions are the same substances that were capable of being used at the aforementioned radiation-resistant resin additives. Accordingly, the foregoing resin compositions may include the aforementioned radiation-resistant resin additives. This being the case, for description of the (a) bisphenol compounds, reference is made to the description of the aforementioned radiation-resistant resin additives.

From the standpoints of radiation resistance and of not decreasing strength/elongation relative to that originally possessed by the resin, it is preferred that the amount of (a) bisphenol compound(s) present within the foregoing resin composition(s) be 0.01 wt % to 10 wt % of the total amount of the resin composition(s), and more preferred that this be 0.1 wt % to 5 wt %.

As (b) amide resin(s) capable of being used at the foregoing resin composition(s), it is sufficient that these be polymer(s) in which amide bonds are present as constituent units. Nylons and other such aliphatic polyamides in which the constituent units include aliphatic skeletons, aramids and other such aromatic polyamides in which the constituent units include aromatic skeletons, polyamide elastomers having hard segments in the form of polyamide blocks and soft segments in the form of polyether, polyester, and/or other such blocks, and so forth may be cited as examples. Thereamong, because they permit more effective manifestation of the radiation resistant function of (a) bisphenol compound(s), polyamide elastomers are suitable. Furthermore, because they impart flexibility and copolymerizability with polyamides, substances having soft segments in the form of polyether blocks are preferred.

As mentioned above, substances having soft segments in the form of polyether blocks are preferred for the foregoing polyamide elastomer. As such polyether blocks, substances having structures derived from at least one species selected from among polyoxyalkylene glycols and (b1) polyether diamines are preferred, and substances having structures derived from polyoxyalkylene glycols or polyether diamines are more preferred.

Polyoxyalkylene glycol, which has a structure resulting from polymerization of alkylene oxide or alkylene glycol, is a polyether diol having a hydroxyl group at either end thereof. As such polyoxyalkylene glycol, substances for which the number of carbons in alkylene group(s) included within the constituent units is not less than 2 but not greater than 4 are, for example, preferred; more specifically, polyethylene glycol, polypropylene glycol, polytetramethylene glyl, and so forth may be cited as examples. From the standpoints of imparting flexibility and copolymerizability with polyamides, it is preferred that the number average molecular weight of polyoxyalkylene glycol be 100 to 2000, and more preferred that this be 200 to 1000.

From the standpoint of copolymerizability with polyamides, it is preferred that the polyether diamine be a polyether that has an amino group at either end thereof. As such polyether diamine, it is for example preferred that this be at least one species indicated by General Formula (5) below.

H₂NR⁹—OₘR¹⁰—NH₂ (5)

At General Formula (5), R⁹ indicates saturated hydrocarbon group(s), each of which independently has not less than 1 carbon, R¹⁰ indicates a saturated hydrocarbon group having not less than 1 carbon and m indicates an integer not less than 1. Furthermore, where there are two or more species of repeating units that contain R⁹, m is the sum of all of the respective repeating units that contain R⁹. Taking the example of General Formula (6) for example, x+y+z=m. From the standpoints of imparting flexibility and copolymerizability, it is preferred that m be not less than 1 but not greater than 200, and it is more preferred that this be not less than 2 but not greater than 100.

As the saturated hydrocarbon groups indicated by R⁹ and R¹⁰ at General Formula (5), while there is no particular limitation with respect thereto so long as the number of carbons is not less than 1, from the standpoint of superior flexibility, it is preferred that the number of carbons be not less than 1 but not greater than 10, and more preferred that this be not less than 2 but not greater than 4. As the structure thereof, this may be either chain-like or ring-like. From the standpoint of chemical interactions with radiation-resistant resin additive(s), a chain-like structure is preferred. Where a chain-like structure is employed, this may be a straight-chain or it may have branched chain(s).

There may be one repeating unit that contains R⁹, or two or more thereof may be present. Where two or more of these repeating units are present, from the standpoint of superior reactivity, it is preferred that the (b1) polyether diamine be at least one species indicated by General Formula (6) below.

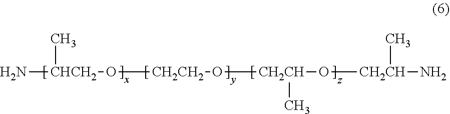

In General Formula (6), x+z indicates an integer not less than 1, and y indicates an integer not less than 1. This will make imparting of flexibility possible. It is preferred that x+z be not less than 1 but not greater than 6, and more preferred that this be not less than 1 but not greater than 4. Furthermore, it is preferred that y be not less than 1 but not greater than 20, and more preferred that this be not less than 1 but not greater than 10. Here, x, y, and z might, for example, be determined by carrying out GPC measurements as at the Examples described below.

As such, polyether diamine indicated by General Formula (6), polyoxyethylene, 1,2-polyoxypropylene, 1,3-polyoxypropylene, or amino-modified polyoxyalkylenes that are copolymers thereof, and other such polyether diamine compounds may be cited as examples. More specifically, the Jeffamine ED series manufactured by Huntsman Corporation of the USA or the like may be favorably employed. Products in the Jeffamine ED series for which, at General Formula (6), x+z is not less than 1 but not greater than 6, and y is not less than 1 but not greater than 20, are ED 600 and ED 900. Thereamong, ED 900 is a substance for which x+z is not less than 1 but not greater than 6, ED 600 is a substance for which x+z is not less than 1 but not greater than 4, ED 900 is a substance for which y is not less than 1 but not greater than 15, and ED 600 is a substance for which y is not less than 1 but not greater than 10.

As the foregoing polyamide elastomer, substances having hard segments in the form of polyamide blocks are preferred. As such polyamide block, from the standpoint of polymerization reactivity, a substance having a structure derived from at least one species of (b2) carboxylic-acid-terminated polyamide is preferred. As such polyamide block capable of making up a hard segment, aliphatic polyamide blocks are preferred; as such aliphatic polyamide block, a substance having structure(s) derived from at least one species of (b21) aminocarboxylic acid indicated by General Formula (3) below (sometimes referred to below as (b21) component), and having structure(s) derived from at least one species of (b22) dicarboxylic acid indicated by General Formula (4) below (sometimes referred to below as (b22) component), is preferred.

HOOC—R⁷NH—CO—R⁷ₙNH₂ (3)

In General Formula (3), R⁷ indicates saturated hydrocarbon group(s), each of which respectively has not less than 1 carbon, and n indicates an integer not less than 0. Furthermore, where there are two or more species of repeating units that contain R⁷, n is the sum of all of the respective repeating units that contain R⁷.

From the standpoints of polymerization reactivity and mechanical properties of the polyamide elastomer that are obtained, it is preferred that n be not less than 1 but not greater than 100, more preferred that this be not less than 10 but not greater than 50, and still more preferred that this be not less than 20 but not greater than 40. Here, n might be determined to be the number average molecular weight as obtained by gel permeation chromatography (GPC).

It is sufficient that such R⁷ making up (b21) component be a saturated hydrocarbon group having not less than 1 carbon. The saturated hydrocarbon group may be of either a chain-like or ring-like structure. From the standpoint of chemical interactions with radiation-resistant resin additive(s), a chain-like structure is preferred. Where a chain-like structure is employed, this may be a straight-chain or it may have branched chain(s). Note, however, that, from the standpoint of polymerization reactivity and mechanical properties of the polyamide elastomer which are obtained, it is preferred that R⁷ be a straight-chain saturated hydrocarbon group having not less than 6 but not greater than 18 carbons. As preferred (b21) component, 1-6 aminohexanoic acid, 1-7 aminoheptanoic acid, 1-8 aminooctanoic acid, 1-9 aminononanoic acid, 1-10 aminodecanoic acid, 1-11 aminoundecanoic acid, 1-12 aminododecanoic acid, 1-14 aminotetradecanoic acid, 1-16 aminohexadecanoic acid, 1-17 aminoheptadecanoic acid, 1-18 aminooctadecanoic acid, and other such aminocarboxylic acids, as well as condensation products thereof, may be cited as examples. Furthermore, where the (b21) component is a condensation product of an aminocarboxylic acid, the condensation product may employ any one of these aminocarboxylic acids, or the condensation product may be such that any two or more of these are used in combination.

Note, however, that there is a tendency for toughness of the polyamide elastomer to increase, and that this occurs in particular with increasing length of the carbon chain(s) at R⁷.

Furthermore, while the (b21) component may be a condensation product of a diamine and a dicarboxylic acid, it being possible to cite nylon 6-6 which is the polycondensation product of hexamethylenediamine and adipic acid, nylon 6-9 which is the polycondensation product of hexamethylenediamine and azelaic acid, nylon 6-10 which is the polycondensation product of hexamethylenediamine and sebacic acid, nylon 6-12 which is the condensation product of hexamethylenediamine and 1-12 dodecanedioic acid, or nylon 9-6 which is the condensation product of nonamethylenediamine and adipic acid and so forth as examples, there is no limitation with respect thereto.

It is preferred that the number average molecular weight (Mn) of the (b21) component be not less than 2000 but not greater than 8000, and it is more preferred that this be not less than 3000 but not greater than 7000. Causing the number average molecular weight to be within such a range will permit attainment of a block copolymer having superior mechanical properties.

Note that the number average molecular weight of the (b21) component might, for example, be calculated using gel permeation chromatography (GPC). Furthermore, where this is the case, it is known that there will be a variation of on the order of 10% in the measurement of number average molecular weight. Accordingly, when the number average molecular weight is to be calculated based on GPC in the context of the present invention, the number average molecular weight is taken to be the average of the results of a plurality of measurement trials. Furthermore, where it is impossible to carry out a plurality of measurement trials, the condition for the number average molecular weight of the (b21) component is taken to be satisfied if the foregoing number average molecular weight falls within such range(s) after allowing for a spread of on the order of 10% in the results obtained from a single measurement trial.

HOOC—R⁸—COOH (4)

In General Formula (4), R⁸ indicates a direct bond or a saturated hydrocarbon group having not less than 1 carbon. The saturated hydrocarbon group may be of either a chain-like or ring-like structure. From the standpoint of chemical interactions with radiation-resistant resin additive(s), a chain-like structure is preferred. Where a chain-like structure is employed, this may be a straight-chain or it may have branched chain(s). As the saturated hydrocarbon group, while there is no particular limitation with respect thereto so long as the number of carbons is not less than 1, from the standpoints of polymerization reactivity and mechanical properties of the polyamide elastomer that are obtained, it is preferred that the number of carbons be not less than 2 but not greater than 10, and still more preferred that this be straight-chain.

As compounds capable of being used as such (b22) component, while oxalic acid, malonic acid, succinic acid, glutaric acid, adipic acid, pimelic acid, suberic acid, azelaic acid, sebacic acid, and other such dicarboxylic acids may be cited as specific examples, there is no limitation with respect thereto. Furthermore, any of such dicarboxylic acids may be used alone, or any two or more thereof may be used in combination.

Where the foregoing (b21) component amino group (A) and (b22) component monocarboxylic acid group (B) are present, while there is no particular limitation with respect to the molar ratio (A/B) thereof, from the standpoint of facilitating attainment of a polyamide elastomer of favorable number average molecular weight, it is preferred that the molar ratio (A/B) be not less than 1/2 but not greater than 5/4, and more preferred that this be substantially 1/1. Here, what is meant by substantially 1/1 is that the number of moles of monocarboxylic acid group and of amino groups as calculated from the weight of raw material are more or less equimolar.

From the standpoint of its role as hard segment in block copolymer, it is preferred that the number average molecular weight (Mn) of (b2) carboxylic-acid-terminated polyamide be not less than 2000, more preferred that this be not less than 4000, still more preferred that this be not less than 2000 but not greater than 8000, and particularly preferred that this be not less than 3000 but not greater than 7000.

As the foregoing polyamide elastomer, from the standpoints of imparting flexibility and copolymerization reactivity, a substance having structure(s) derived from at least one species of (b3) diamine indicated by General Formula (7) below, (sometimes referred to below as (b3) component) and having structure(s) derived from at least one species of (b2) carboxylic-acid-terminated polyamide as hard segment polyamide block is more preferred.

H₂N—R¹¹—NH₂ (7)

In General Formula (7), R¹¹ indicates a saturated hydrocarbon group having not less than 1 carbon. As R¹¹, while there is no limitation with respect thereto so long as it is a straight-chain or branched saturated hydrocarbon group having not less than 1 carbon, from the standpoint of further improving the mechanical properties of the polyamide elastomer that is obtained, it is preferred that the number of carbons be not less than 2 but not greater than 14, and more preferred that this be not less than 4 but not greater than 12. While ethylenediamine, trimethylenediamine, tetramethylenediamine, pentamethylenediamine, hexamethylenediamine, heptamethylenediamine, octamethylenediamine, nonamethylenediamine, decamethylenediamine, undecamethylenediamine, dodecamethylenediamine, 2,2-4/2,4,4-trimethylhexamethylenediamine, 3-methylpentamethyldiamine, and other such aliphatic diamines may be cited as specific examples, there is no limitation with respect thereto. Furthermore, thereamong, from the aforementioned standpoint, at least one species of aliphatic diamine selected from among hexamethylenediamine, heptamethylenediamine, octamethylenediamine, nonamethylenediamine, decamethylenediamine, undecamethylenediamine, and dodecamethylenediamine is more preferred.

Where structure(s) derived from (b3) component are present in the foregoing (b) amide resin(s), there is no particular limitation with respect to the molar ratio in such structural unit(s) and structural unit(s) derived from (b1) component. Flexibility may be increased by increasing the molar ratio of structure(s) derived from (b1) component. Accordingly, this may be chosen as appropriate in correspondence to the intended usage thereof.

As the foregoing polyamide elastomer, from the standpoint of imparting flexibility, a substance having structure(s) derived from at least one species selected from among polyoxyalkylene glycols and (b1) polyether diamines as soft segment, and having structure(s) derived from at least one species of (b2) carboxylic-acid-terminated polyamide as hard segment, is more preferred. Furthermore, from the standpoint of copolymerization reactivity, a substance having structure(s) derived from at least one species of (b3) diamine indicated by General Formula (7) and structure(s) derived from at least one species of (b2) carboxylic-acid-terminated polyamide as hard segment, and structure(s) derived from at least one species selected from among polyoxyalkylene glycols and (b1) polyether diamines as soft segment, is still more preferred. Furthermore, as structure derived from (b2) carboxylic-acid-terminated polyamide, substances having structure(s) derived from (b21) component and structure(s) derived from (b22) component are particularly preferred.

It is preferred that the melt viscosity (melt flow rate; MFR) of the foregoing polyamide elastomer be 0.1 to 20 (g/10 min) at 230° C. and 2.16 kgf (21.2 N). This will permit attainment of satisfactory extrusion molding characteristics. To cause melt viscosity to be within such range, reaction temperature, reaction time, solution concentration, and so forth at the time of polymerization should be chosen as appropriate.

It is preferred that the Shore D hardness of the foregoing polyamide elastomer be 50 to 100, and more preferred that this be 60 to 80. This will permit attainment of flexibility in the molded article. To cause Shore D hardness to be within such range, the amount of (b1) component loaded therein, and where (b3) component is employed, the ratio between the (b1) component and the (b3) component that are loaded therein, should be chosen as appropriate.

It is preferred that the number average molecular weight of the foregoing polyamide elastomer be not less than 10000 but not greater than 150000, and it is more preferred that this be not less than 20000 but not greater than 100000. Causing the number average molecular weight to be within such range will permit superior attainment of mechanical properties and workability.

It is preferred that the foregoing polyamide elastomer be such that the percent elongation at fracture as measured during tensile testing of the molded article be not less than 100% but not greater than 600%, and more preferred that this be not less than 200% but not greater than 600%. Furthermore, it is preferred that stress at fracture be not less than 20 MPa but not greater than 100 MPa, and more preferred that this be not less than 30 MPa but not greater than 90 MPa. Note that tensile testing might, for example, be carried out in accordance with the method which is described below. Alternatively, this might be carried out in accordance with JIS K 7161.

The foregoing polyamide elastomer may contain phosphorous compound(s).

This will make it possible to further improve stress at fracture and/or percent elongation at fracture of the molded article. This being the case, it might, for example, be suitable for use as a medical balloon. Furthermore, it may prevent coloration due to oxidation and/or stabilize polymerization reactivity during operations for manufacture of the polyamide elastomer as described below. As such phosphorous compounds, phosphoric acid, pyrophosphoric acid, polyphosphoric acid, phosphorous acid, hypophosphorous acid, as well as alkali metal salts and alkaline earth metal salts thereof, and so forth may be cited as examples. Thereamong, from the standpoints of improving stabilization of polymerization reactivity, imparting stability in heat resistance to the polyamide elastomer, and improving mechanical properties of the molded article, phosphorous acid and hypophosphorous acid, as well as the alkali metal salts and alkaline earth metal salts thereof, are preferred.

It is preferred that phosphorous compound(s) be present in such an amount that elemental phosphorous within the polyamide resin composition is not less than 5 ppm but not greater than 5000 ppm, more preferred that this be not less than 20 ppm but not greater than 4000 ppm, and still more preferred that this be not less than 30 ppm but not greater than 3000 ppm.

Besides the aforementioned (a) bisphenol compound(s) and phosphorous compound(s), any of various additives may, in correspondence to purpose, be blended within the foregoing polyamide elastomer within such range(s) as will not impair the properties thereof. More specifically, a heat-resistant agent, ultraviolet light absorber, photostabilizer, antioxidant, antistatic agent, lubricant, slip agent, nucleating agent, tackifier, mold release agent, plasticizer, pigment, dye, flame retardant, stiffener, inorganic filler, microfilament, radiopaque agent, and so forth may be added thereto.

Below, where the (b) amide resin at the foregoing radiation-resistant medical polyamide resin composition is a polyamide elastomer having a structure derived from (b1) component, (b21) component, and (b22) component, or from (b1) component, (b21) component, (b22) component, and (b3) component, embodiments of methods for manufacturing the polyamide elastomer are described.

The foregoing polyamide elastomer may be obtained by causing reaction of at least the (b21), (b22), and (b1) components, and optionally also of the (b3) component which may be employed as necessary. Methods in which the (b21), (b22), and (b1) components, or the (b21), (b22), (b1), and (b3) components, are simultaneously mixed and reacted, methods in which the (b21) component and the (b22) component are reacted and the remaining component(s) are thereafter added and reacted, and so forth may be cited as examples. Thereamong, from the standpoint of efficient synthesis of block copolymer(s) having desired hard segment(s) and soft segment(s), it is preferred that this be obtained by a manufacturing method comprising (i) a step in which (b21) component and (b22) component are mixed and reacted to obtain a prepolymer (hereinafter referred to as “step (i)”), and a step in which (b1) component, or (b1) component and (b3) component, are mixed and reacted with the prepolymer obtained at step (i) (hereinafter referred to as “step (ii)”).

While there is no particular limitation with respect to the mixture ratio employed at the time that (b21) component and (b22) component are mixed at step (i), from the standpoint of facilitating attainment of desired hard segment length, it is preferred that the molar ratio (A/B) between (b21) component amino groups (A) and (b22) component monocarboxylic acid groups (B) be not less than 1/2 but not greater than 5/4, and more preferred that this be substantially 1/1. Note that even at manufacturing methods in which (b1), (b21), and (b22) components, or (b1), (b21), (b22), and (b3) components, are simultaneously mixed and reacted, and even at manufacturing methods comprising step (i) and step (ii), it is preferred that these be mixed therein in such fashion as to cause amino groups and carboxylic acid groups at all components, i.e., all of the (b1), (b21), and (b22) components, or all of the (b1), (b21), (b22), and (b3) components, to be substantially equimolar.

Furthermore, it is desirable that addition of compounds capable of disturbing the equimolarity of the amino groups and the carboxylic acid groups be done only to such degree as will not result in lowering of the desired properties.

While there is no particular limitation with respect to the mixture ratio of the respective components (b1), (b21), and (b22), it is preferred that the (b21) component be 70 wt % to 98.5 wt % of the total of the (b1), (b21), and (b22) components, and more preferred that this be 85 wt % to 98 wt % thereof. It is preferred that the (b22) component be 0.5 wt % to 20 wt % of the total of the (b1), (b21), and (b22) components, and more preferred that this be 1 wt % to 10 wt % thereof. It is preferred that the (b1) component be 0.5 wt % to 20 wt % of the total of the (b1), (b21), and (b22) components, and more preferred that this be 1 wt % to 10 wt % thereof. Furthermore, where (b3) component is employed, while there is no particular limitation with respect to the mixture ratio of the respective components (b1), (b21), (b22), and (b3), it is preferred that the (b21) component be 70 wt % to 98.5 wt % of the total of the (b1), (b21), (b22), and (b3) components, and more preferred that this be 85 wt % to 98 wt % thereof. It is preferred that the (b22) component be 0.5 wt % to 20 wt % of the total of the (b1), (b21), (b22), and (b3) components, and more preferred that this be 1 wt % to 10 wt % thereof. It is preferred that the (b1) component be 0.5% to 20% of the total of the (b1), (b21), (b22), and (b3) components, and more preferred that this be 1 wt % to 10 wt % thereof. It is preferred that the (b3) component be 0.5 wt % to 30 wt % of the total of the (b1), (b21), (b22), and (b3) components, and more preferred that this be 1 wt % to 20 wt % thereof.

Step (ii) should therefore be taken into consideration when determining the amounts of (b21) component and (b22) component to be mixed at step (i). Note, however, that, it being preferred that the molar ratio of amino groups and carboxylic acid groups at all components, i.e., all of the (b1), (b21), and (b22) components, or all of the (b1), (b21), (b22), and (b3) components, be taken into consideration as has been mentioned above, it is preferred that the amounts of (b21) component and (b22) component to be mixed are determined in such fashion as to cause the molar ratio to be substantially equimolar. Furthermore, where the (b21) component is a polycondensation product, the amounts of (b21) component and (b22) component to be mixed may also be determined based on the pre-polymerization compounds.

In methods for manufacturing polyamide elastomer in accordance with the present invention, the reactions of steps (i) and (ii) may be carried out in the presence of solvent, or absence of solvent. Since there is no need for purification or the like, and thus the polyamide elastomer can be easily obtained, it is preferred that the reactions be carried out in the absence of solvent, i.e., without the use of solvent. Such reactions in the absence of solvent may be carried out using the melt knead method. It is therefore preferred that reaction of the (b1) component, or the (b1) component and the (b3) component, with the (b21) component and the (b22) component at step (i), as well as the prepolymer at step (ii), be carried out using the melt knead method.

In the reaction for polymerization of the (b1), (b21), and (b22) components, or the (b1), (b21), (b22), and (b3) components, the normal-pressure melt polycondensation reaction or the vacuum melt polycondensation reaction, or a combination thereof, may be employed. Where vacuum melt polycondensation is employed, from the standpoint of polymerization reactivity, it is preferred that this be carried out in a nitrogen gas environment, and that the pressure inside the reaction vessel be 0.1 to 0.01 (MPa). These melt polycondensation reactions may be carried out using the melt knead method in the absence of solvent.

While there is no particular limitation with respect to the reaction temperature(s) employed at step (i) and step (ii) in methods for manufacturing the foregoing polyamide elastomer, so long as the polymerization reaction takes place, from the standpoint of achieving balance between suppression of pyrolysis and reaction rate, it is preferred that this be 160° to 300° C., and more preferred that this be carried out at 200° to 280° C. Note that the reaction temperatures employed at step (i) and step (ii) may be the same or they may be different.

From the standpoints of suppressing coloration, increasing in molecular weight, and so forth, it is preferred that the polymerization reaction time(s) employed at step (i) and step (ii) in methods for manufacturing the foregoing polyamide elastomer be 3 to 10 hours. Note that the polymerization reaction times employed at step (i) and step (ii) may be the same or they may be different.

The foregoing polyamide elastomer manufacturing method may be carried out in batch fashion or in continuous fashion. For example, this may be carried out in batch fashion using a batch-type reaction tank or the like, or it may be carried out in continuous fashion using a single-tank or multiple-tank continuous reactor, tubular continuous reactor, and/or the like, alone or in combination.

During manufacture of the foregoing polyamide elastomer, phosphorous compound(s) may, where necessary, be used as catalyst. As such phosphorous compounds, phosphoric acid, pyrophosphoric acid, polyphosphoric acid, phosphorous acid, hypophosphorous acid, as well as alkali metal salts and alkaline earth metal salts thereof, and so forth may be cited as examples. Thereamong, from the standpoints of improving stabilization of polymerization reactivity, imparting stability in heat resistance to the polyamide elastomer, and improving mechanical properties of the molded article, employment of phosphorous acid and hypophosphorous acid, as well as the alkali metal salts and alkaline earth metal salts thereof, and/or other such inorganic phosphorous compounds, is preferred.

It is preferred that the weight, upon preparation, of such phosphorous compound(s) with respect to the total weight of the (b1), (b21), and (b22) components, or where (b3) component is employed, of the (b1), (b21), (b22), and (b3) components, at least one of step (i) and step (ii), be not less than 10 ppm but not greater than 10000 ppm, and more preferred that this be not less than 100 ppm but not greater than 5000 ppm. Furthermore, at such time, where the (b21) component is a polycondensation product, this may be carried out based on amount(s) of pre-polycondensation compound(s) to be added. Note that because phosphorous compound(s) may be discharged outside the reaction system due to byproducts generated as a result of reaction, the weight of the phosphorous compounds upon preparation and the amount of elemental phosphorous that is present in the polyamide elastomer need not be the same. It is preferred that the amount of elemental phosphorous present in the polyamide elastomer that is obtained be not less than 5 ppm but not greater than 5000 ppm, more preferred that this be not less than 20 ppm but not greater than 4000 ppm, and still more preferred that this be not less than 30 ppm but not greater than 3000 ppm.

Following reaction of the respective components at step (ii), the polymer might, for example, in its molten state, be drawn out therefrom in string-like fashion and cooled to obtain product in the form of pellets or the like as necessary.

The foregoing radiation-resistant medical polyamide resin composition might, for example, be obtained by (I) a method in which (b) amide resin(s) is synthesized in the presence of (a) bisphenol compound(s), other additive(s) being mixed therewith as necessary, (II) a method in which (a) bisphenol compound(s), previously synthesized (b) amide resin(s), and where necessary other additive(s) that are to be employed, are mixed, and so forth. Method (I) tends to permit (a) bisphenol compound(s) to be more homogeneously dispersed throughout (b) amide resin(s) than is the case with method (II). For method (I), taking the example of the aforementioned embodiment of a polyamide elastomer manufacturing method, it will be sufficient if (a) bisphenol compound(s) are made to be present during reaction of at least the (b21), (b22), and (b1) components, and optionally also of the (b3) component which may be employed as necessary. There is no particular limitation with respect to the manner in which (a) bisphenol compound(s) are added thereto, it being possible for the necessary amount to be added all at once, or for this to be divided into multiple portions that are added at different times. With regard to the mixing method that is employed when the respective components are mixed at method (I) and method (II), it is sufficient that this be capable of causing mixing such as will permit other additive(s) to attain a homogeneous concentration distribution within (b) amide resin(s) in the case of method (I), and such as will permit (a) bisphenol compound(s) and so forth to attain a homogeneous concentration distribution within (b) amide resin(s) in the case of method (II). Methods employing tumbling mixer(s), ribbon blender(s), Henschel mixer(s), and/or other such mixer(s), open roller(s), kneader(s), Banbury mixer(s), continuous mixer(s), single-screw extruder(s) (single-shaft extruder(s)), multi-screw extruder(s) (multi-shaft extruder(s)) having two or more shafts, and/or other such kneaders, may be cited as examples. Thereamong, from the standpoint of mixing efficiency, single-screw extruders and multi-screw extruders are preferred. Regarding a multi-screw extruder, while this may be intermeshing or non-intermeshing, intermeshing is preferred; with respect to intermeshing, while this may be corotating or counterrotating, counterrotating is preferred. Mixing conditions may be chosen as appropriate in correspondence to amide resin properties.

Where necessary, the respective components may be made to undergo drying processing before the aforementioned mixer and/or kneader is used to carry out mixing. Causing moisture content within the resin composition to be 100 ppm to 3000 ppm will prevent occurrence of bubbles due to steam in the molded article. As drying conditions at such time, 60° to 100° C. for 4 hours to 12 hours is preferred.

The form of the foregoing resin composition may be chosen as appropriate in correspondence to the intended usage thereof, it being possible to cite powder form, pellet form, and so forth as examples.

Radiation-Resistant Medical Molded Article

The foregoing radiation-resistant medical molded article is fabricated using the aforementioned resin composition. Using the aforementioned resin composition makes it possible to obtain a molded article having superior radiation-resistance. It is therefore suitable where gamma rays, electron beams, and/or other such radiation is employed, and it is in particular suitable for medical use where sterilization processing is carried out by means of high-intensity electron beams. Furthermore, as molded articles for medical use, it is in particular suitably employed for medical tubes and medical balloons, because it can be expected that these will undergo sterilization processing by means of high-intensity electron beams. Furthermore, the foregoing radiation-resistant medical molded article may be molded using the aforementioned resin composition in any of the various conventional molding methods, such as extrusion molding, injection molding, blowing molding, and so forth, in correspondence to intended usage thereof and so forth.

Furthermore, where (b) amide resin(s) is/are prescribed polyamide elastomer(s), because polyether chain(s) and/or polyamide chain(s) will be present to an appropriate degree, there will be little change in properties due to hygroscopicity, and resin melt characteristics will cause extrusion molding characteristics and pultrusion molding characteristics to be superior, and blow molding characteristics will be superior, and toughness will be superior. For this reason, it will, for example, be suitable as a constituent material for extrusion-molded medical tubing and the like, blow-molded medical bottles, medical balloons, and/or other such members.

### EXAMPLES

Although the present invention is described more specifically in terms of examples below, the technical scope of the present invention is not to be limited by these examples.

1. Pellet Fabrication Method

After dry-blending the respective polyamide resins and additives employed in the examples and comparative examples, a ϕ16 mm (L/D=40) corotating intermeshing two-shaft extruder was used to carry out melt-kneading at a setpoint temperature of 200° to 230° C. and a screw rotational speed of 600 rpm to obtain a polyamide resin composition in pellet form.

2. Tubing Fabrication Method

The aforementioned polyamide resin compositions in pellet form is subjected to extrusion molding, using a single-shaft extruder (ϕ15 mm; L/D=28) at a setpoint temperature of 200° to 230° C. and a screw rotational speed of 20 to 30 (rpm), to fabricate molded tubing having an outside diameter of 0.88 (mm) and an inside diameter 0.46 (mm). The tubing which was obtained was used as samples for the tensile testing and electron irradiation testing described below.

3. Electron Beam Irradiation Testing

An electron beam irradiation apparatus (Dynamitron Electron Accelerator; manufactured by RDI) was used to carry out electron beam irradiation testing under a condition of a surface expected dose of 80 kGy (acceleration voltage 4.8 (MV); electric current 20 mA; processing speed 6.2 m/min), by using dose reader (UV-1800 Spectrophotometer for CTA dosimeter manufactured by Shimadzu Corporation), and CTA dosimeter (FTR-125 manufactured by Fuji Photo Film Co., Ltd.),

The method employed for testing was such that samples were evenly arrayed on the horizontal surface of an irradiation cart on which supports (5 sheets of cardboard) had been placed, and electron beam irradiation was carried out from above in the vertical direction.

4. Tensile Testing

Tensile testing was carried out in a constant-temperature phase at a temperature of 23° C. using a Model 5564 manufactured by Instron. Test conditions were such that chuck separation was 50 mm and elongation rate was 200 (mm/min). Drying of samples was carried out by using a vacuum dryer to carry out drying for 4 hours under vacuum conditions of −0.1 (MPa).

### Manufacturing Example

1200 g of 12-aminododecanoic acid and 0.6 g of hypophosphorous acid were placed in a reaction vessel of volume 3 L equipped with an agitator, temperature controller, pressure gauge, nitrogen gas inlet port, and condensate discharge port, the interior of the vessel was sufficiently replaced with nitrogen, temperature was increased to 280° C. for 1 hour to cause melting, and polymerization was carried out until number average molecular weight was 6200 to obtain (b21) component making up a hard segment.

In addition, 35 g (0.24 mol) of adipic acid ((b22) component), which was equimolar with respect to the molar amount of terminal amine groups at the (b21) component that was obtained, was added, and the reaction was carried out for 1 hour at 220° C. to obtain carboxylic-acid-terminated polyamide (b2). Number average molecular weight of the carboxylic-acid-terminated polyamide (b2) was 6500.

To achieve equimolarity with respect to the carboxylic acid groups at either end of the carboxylic-acid-terminated polyamide (b2) that was obtained, 14 g (0.12 mol) of hexamethylenediamine serving as the (b3) component, and 72 g (0.12 mol) of polyether diamine (Jeffamine ED 600; manufactured by Huntsman Corporation) serving as the (b1) component were placed therein, temperature was increased to 260° C., and polymerization was carried out for a further 4 hours to obtain polyamide elastomer (HMDP6 (55)).

Following termination of polymerization, agitation was stopped, and the polymer was extracted in string-like fashion from an access port in a molten state as a colorless transparent polyamide elastomer, which was cooled with water and thereafter pelletized to obtain approximately 1 kg of pellets.

### Example 1

5 parts by weight of bisphenol compound (Yoshinox BB; manufactured by Mitsubishi Chemical Corporation) was dry-blended with 95 parts by weight of pellets of polyamide elastomer (Pebax 7233; manufactured by Arkema K.K.) having a structure derived from polytetramethylene glycol as the soft segment and having a structure derived from polyamide 12 as the hard segment, and these were mixed using a two-shaft extruder to obtain semitransparent colorless pellets that were roughly 3 mm in diameter and 3 mm in length. Gel permeation chromatography (GPC) was used to confirm that there was no decrease in molecular weight at this time. The pellets obtained were thereafter dried for 6 hours at 80° C. to cause the moisture content to be 910 ppm. In addition, a single-shaft extruder was used to obtain hollow tubing of outside diameter 0.88 mm and inside diameter 0.46 mm. As a result of performing tensile testing on 10 samples of this tubing, the average value for percent elongation at fracture was found to be 399.1%, and the average value for load at fracture was found to be 31.1 N.

This tubing was subjected to 80 kGy of electron beam irradiation, and as a result of tensile testing performed in similar fashion 24 hours thereafter, it was found that the average value for percent elongation at fracture was 401.2%, and the average value for load at fracture was 30.3 N.

### Example 2

Except for the fact that 1 part by weight of bisphenol compound (Yoshinox BB) was used with respect to 99 parts by weight of pellets of polyamide elastomer (Pebax 7233), the same procedure as in Example 1 was employed to obtain semitransparent colorless pellets. GPC was used to confirm that there was no decrease in molecular weight at this time. The pellets obtained were thereafter dried for 6 hours at 80° C. to cause moisture content to be 860 ppm. In addition, a single-shaft extruder was used to obtain hollow tubing of outside diameter 0.88 mm and inside diameter 0.46 mm. As a result of performing tensile testing on 10 samples of this tubing, the average value for percent elongation at fracture was found to be 412.1%, and the average value for load at fracture was found to be 34.6 N.

This tubing was subjected to 80 kGy of electron beam irradiation, and as a result of tensile testing performed in similar fashion 24 hours thereafter, it was found that the average value for percent elongation at fracture was 413.6%, and the average value for load at fracture was 32.9 N.

### Example 3

Except for the fact that 0.5 part by weight of bisphenol compound (Yoshinox BB) was used with respect to 99.5 parts by weight of pellets of polyamide elastomer (Pebax 7233), the same procedure as in Example 1 was employed to obtain semitransparent colorless pellets. GPC was used to confirm that there was no decrease in molecular weight at this time. The pellets obtained were thereafter dried for 6 hours at 80° C. to cause the moisture content to be 780 ppm. In addition, a single-shaft extruder was used to obtain hollow tubing of outside diameter 0.88 min and inside diameter 0.46 mm. As a result of performing tensile testing on 10 samples of this tubing, the average value for percent elongation at fracture was found to be 387%, and the average value for load at fracture was found to be 33.1 N.

This tubing was subjected to 80 kGy of electron beam irradiation, and as a result of tensile testing performed in similar fashion 24 hours thereafter, it was found that the average value for percent elongation at fracture was 390.4%, and the average value for load at fracture was 31.9 N.

### Example 4

Except for the fact that 0.1 part by weight of bisphenol compound (Yoshinox BB) was used with respect to 99.9 parts by weight of pellets of polyamide elastomer (Pebax 7233), the same procedure as in Example 1 was employed to obtain semitransparent colorless pellets. GPC was used to confirm that there was no decrease in molecular weight at this time. The pellets obtained were thereafter dried for 6 hours at 80° C. to cause the moisture content to be 820 ppm. In addition, a single-shaft extruder was used to obtain hollow tubing of outside diameter 0.88 mm and inside diameter 0.46 mm. As a result of performing tensile testing on 10 samples of this tubing, the average value for percent elongation at fracture was found to be 410.7%, and the average value for load at fracture was found to be 33.5 N.

This tubing was subjected to 80 kGy of electron beam irradiation, and as a result of tensile testing performed in similar fashion 24 hours thereafter, it was found that the average value for percent elongation at fracture was 414.2%, and the average value for load at fracture was 32.4 N.

### Example 5

1 part by weight of bisphenol compound (Yoshinox 425; manufactured by Mitsubishi Chemical Corporation) was dry-blended with 99 parts by weight of pellets of polyamide elastomer (Pebax 7233), and these were mixed using a two-shaft extruder to obtain semitransparent colorless pellets that were roughly 3 mm in diameter and 3 mm in length. GPC was used to confirm that there was no decrease in molecular weight at this time. The pellets obtained were thereafter dried for 6 hours at 80° C. to cause the moisture content to be 830 ppm. In addition, a single-shaft extruder was used to obtain hollow tubing of outside diameter 0.88 mm and inside diameter 0.46 mm. As a result of performing tensile testing on 10 samples of this tubing, the average value for percent elongation at fracture was found to be 389.6%, and the average value for load at fracture was found to be 32.8 N.

This tubing was subjected to 80 kGy of electron beam irradiation, and as a result of tensile testing performed in similar fashion 24 hours thereafter, it was found that the average value for percent elongation at fracture was 390.7%, and the average value for load at fracture was 31.6 N.

### Example 6

1 part by weight of bisphenol compound (Yoshinox BB) was dry-blended with 99 parts by weight of pellets of polyamide elastomer obtained at the Manufacturing Example, and these were mixed using a two-shaft extruder to obtain semitransparent colorless pellets of such shape that they were roughly 3 mm in diameter and 3 mm in length. GPC was used to confirm that there was no decrease in molecular weight at this time. The pellets obtained were thereafter dried for 6 hours at 80° C. to cause the moisture content to be 790 ppm. In addition, a single-shaft extruder was used to obtain hollow tubing of outside diameter 0.88 mm and inside diameter 0.46 mm. As a result of performing tensile testing on 10 samples of this tubing, the average value for percent elongation at fracture was found to be 400.5%, and the average value for load at fracture was found to be 30.9 N.

This tubing was subjected to 80 kGy of electron beam irradiation, and as a result of tensile testing performed in similar fashion 24 hours thereafter, it was found that the average value for percent elongation at fracture was 401.5%, and the average value for load at fracture was 31.5 N.

### Example 7

0.5 parts by weight of bisphenol compound (Yoshinox BB) was dry-blended with 99.5 parts by weight of pellets of polyamide elastomer obtained at the Manufacturing Example, and these were mixed using a two-shaft extruder to obtain semitransparent colorless pellets of such shape that they were roughly 3 mm in diameter and 3 mm in length. GPC was used to confirm that there was no decrease in molecular weight at this time. The pellets obtained were thereafter dried for 6 hours at 80° C. to cause the moisture content to be 890 ppm. In addition, a single-shaft extruder was used to obtain hollow tubing of outside diameter 0.88 mm and inside diameter 0.46 mm. As a result of performing tensile testing on 10 samples of this tubing, the average value for percent elongation at fracture was found to be 404.1%, and the average value for load at fracture was found to be 32.4 N.

This tubing was subjected to 80 kGy of electron beam irradiation, and as a result of tensile testing performed in similar fashion 24 hours thereafter, it was found that the average value for percent elongation at fracture was 403.7%, and the average value for load at fracture was 32.2 N.

### Comparative Example 1

0.5 parts by weight of additive a (Irganox 1010; manufactured by BASF Corporation) and 0.5 parts by weight of additive b (Irganox 1098; manufactured by BASF Corporation) were dry-blended with 99 parts by weight of pellets of polyamide elastomer (Pebax 7233), and these were mixed using a two-shaft extruder to obtain semitransparent colorless pellets that were roughly 3 mm in diameter and 3 mm in length. GPC was used to confirm that there was no decrease in molecular weight at this time. The pellets obtained were thereafter dried for 6 hours at 80° C. to cause the moisture content to be 800 ppm. In addition, a single-shaft extruder was used to obtain hollow tubing of outside diameter 0.88 mm and inside diameter 0.46 mm. As a result of performing tensile testing on 10 samples of this tubing, the average value for percent elongation at fracture was found to be 389.9%, and the average value for load at fracture was found to be 28.6 N.

This tubing was subjected to 80 kGy of electron beam irradiation, and as a result of tensile testing performed in similar fashion 24 hours thereafter, it was found that the average value for percent elongation at fracture was 413.2%, and the average value for load at fracture was 25.1 N.

### Comparative Example 2

The pellets of polyamide elastomer obtained at the Manufacturing Example were dried for 6 hours at 80° C. to cause the moisture content to be 830 ppm. In addition, a single-shaft extruder was used to obtain hollow tubing of outside diameter 0.88 mm and inside diameter 0.46 mm. As a result of performing tensile testing on 10 samples of this tubing, the average value for percent elongation at fracture was found to be 410.8%, and the average value for load at fracture was found to be 32.5 N.

This tubing was subjected to 80 kGy of electron beam irradiation, and as a result of tensile testing performed in similar fashion 24 hours thereafter, it was found that the average value for percent elongation at fracture was 436.3%, and the average value for load at fracture was 30.0 N.

### Comparative Example 3

1 part by weight of additive b (Irganox 1098) was dry-blended with 99 parts by weight of pellets of polyamide elastomer obtained at the Manufacturing Example, and these were mixed using a two-shaft extruder to obtain semitransparent colorless pellets that were roughly 3 mm in diameter and 3 mm in length. GPC was used to confirm that there was no decrease in molecular weight at this time. The pellets obtained were thereafter dried for 6 hours at 80° C. to cause the moisture content to be 760 ppm. In addition, a single-shaft extruder was used to obtain hollow tubing of outside diameter 0.88 mm and inside diameter 0.46 mm. As a result of performing tensile testing on 10 samples of this tubing, the average value for percent elongation at fracture was found to be 449.7%, and the average value for load at fracture was found to be 35.7 N.

This tubing was subjected to 80 kGy of electron beam irradiation, and as a result of tensile testing performed in similar fashion 24 hours thereafter, it was found that the average value for percent elongation at fracture was 473.9%, and the average value for load at fracture was 32.9 N.

Compositions and measurements results for Examples 1-7 and Comparative Examples 1-3 are shown at TABLE 1.

TABLE 1
Compositions and Measurement Results

| | | | | | | | | | | Compar- | Compar- | Compar- |
|---|---|---|---|---|---|---|---|---|---|---|---|---|
| | | | | | | | | | | ative | ative | ative |
| | | | Example | Example | Example | Example | Example | Example | Example | Example | Example | Example |
| | | | 1 | 2 | 3 | 4 | 5 | 6 | 7 | 1 | 2 | 3 |
| PAE* | Pebax 7233 | [parts by | 95 | 99 | 99.5 | 99.9 | 99 | | | 99 | | |
| | | weight] | | | | | | | | | | |
| | HMDP(55) | [parts by | | | | | | 99 | 99.5 | | 100 | 99 |
| | | weight] | | | | | | | | | | |
| Additive | Yoshinox BB | [parts by | 5 | 1 | 0.5 | 0.1 | | 1 | 0.5 | | | |
| | | weight] | | | | | | | | | | |
| | Yoshinox 425 | [parts by | | | | | 1 | | | | | |
| | | weight] | | | | | | | | | | |
| | Irganox 1010 | [parts by | | | | | | | | 0.5 | | |
| | | weight] | | | | | | | | | | |
| | Irganox 1098 | [parts by | | | | | | | | 0.5 | | 1 |
| | | weight] | | | | | | | | | | |
| Before | Percent | [%] | 399.1 | 412.1 | 387 | 410.7 | 389.6 | 400.5 | 404.1 | 389.9 | 410.8 | 449.7 |
| irradiation | elongation | | | | | | | | | | | |
| | at fracture** | | | | | | | | | | | |
| | Load at | [N] | 31.1 | 34.6 | 33.1 | 33.5 | 32.8 | 30.9 | 32.4 | 28.6 | 32.5 | 35.7 |
| | fracture** | | | | | | | | | | | |
| After | Percent | [%] | 401.2 | 413.6 | 390.4 | 414.2 | 390.7 | 401.5 | 403.7 | 413.2 | 436.3 | 473.9 |
| irradiation | elongation | | | | | | | | | | | |
| | at fracture** | | | | | | | | | | | |
| | Load at | [N] | 30.3 | 32.9 | 31.9 | 32.4 | 31.6 | 31.5 | 32.2 | 25.1 | 30.0 | 32.9 |
| | fracture** | | | | | | | | | | | |

*Polyamide elastomer
**Average value over n = 10 trials

From the Examples and Comparative Examples, it is clear that employment of bisphenol compound(s) of prescribed structure(s) makes it possible to suppress deterioration in load at fracture and percent elongation at fracture that would otherwise occur due to irradiation with radiation.

1. A radiation-resistant medical polyamide resin composition comprising:
(a) a bisphenol compound indicated by General Formula (1) below, or General Formula (2) below; and
(b) an amide resin; 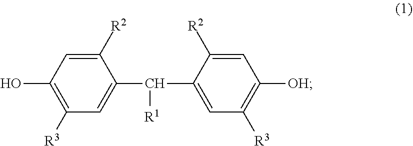
wherein, R¹, R², and R³ respectively may be the same or different and indicate a hydrogen atom or a saturated hydrocarbon group having not less than 1 carbon; 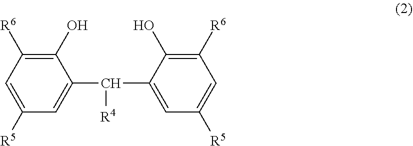
wherein R⁴, R⁵, and R⁶ respectively may be the same or different and indicate a hydrogen atom or a saturated hydrocarbon group having not less than 1 carbon.

2. The radiation-resistant medical polyamide resin composition according to claim 1, wherein the (a) bisphenol compound is present therein in an amount that is 0.01 wt % to 10 wt %.

3. The radiation-resistant medical polyamide resin composition according to claim 1, wherein the (a) bisphenol compound is 4,4′-butylidenebis-(6-t-butyl-3-methylphenol) or 2,2′-methylenebis-(4-ethyl-6-t-butylphenol).

4. The radiation-resistant medical polyamide resin composition according to claim 1, wherein the (b) amide resin
has a structure derived from at least one species selected from among polyoxyalkylene glycol and (b1) polyether diamine as soft segment, and
has a structure derived from at least one species of (b2) carboxylic-acid-terminated polyamide as hard segment.

5. The radiation-resistant medical polyamide resin composition according to claim 4, wherein
the (b2) carboxylic-acid-terminated polyamide
has a structure derived from at least one species of (b21) aminocarboxylic acid indicated by General Formula (3) below, and
has a structure derived from at least one species of (b22) dicarboxylic acid indicated by General Formula (4) below;
HOOC—R⁷NH—CO—R⁷ₙNH₂ (3)
wherein R⁷ indicates saturated hydrocarbon groups, each of which independently has not less than 1 carbon,
n indicates an integer not less than 0, and
when there are two or more species of repeating units that contain R⁷, n is the sum of all of the respective repeating units that contain R⁷;
HOOC—R⁸—COOH (4);
and
wherein R⁸ indicates a direct bond or a saturated hydrocarbon group having not less than 1 carbon.

6. The radiation-resistant medical polyamide resin composition according to claim 4, wherein
the (b1) polyether diamine is at least one species indicated by General Formula (5) below;
H₂NR⁹—OₘR¹⁰—NH₂ (5)
wherein R⁹ independently indicates saturated hydrocarbon groups, each of which has not less than 1 carbon,
R¹⁰ indicates a saturated hydrocarbon group having not less than 1 carbon,
m indicates an integer not less than 1, and
when there are two or more species of repeating units that contain R⁹, m is the sum of all of the repeating units each of which contains R⁹.

7. The radiation-resistant medical polyamide resin composition according to claim 4, wherein
the (b1) polyether diamine is at least one species indicated by General Formula (6) below; 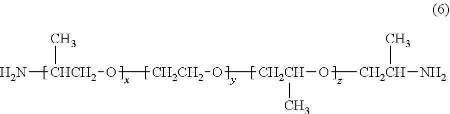
wherein x+z indicates an integer not less than 1, and
y indicates an integer not less than 1.

8. The radiation-resistant medical polyamide resin composition according to claim 4, wherein
the (b) amide resin has: a structure derived from at least one species selected from among the polyoxyalkylene glycol and the (b1) polyether diamine; a structure derived from at least one species of the (b2) carboxylic-acid-terminated polyamide; and a structure derived from at least one species of (b3) diamine indicated by General Formula (7) below;
H₂N—R¹¹—NH₂ (7)
wherein R¹¹ indicates the saturated hydrocarbon group having not less than 1 carbon.

9. The radiation-resistant medical polyamide resin composition according to claim 4, wherein number average molecular weight (Mn) of the (b2) carboxylic-acid-terminated polyamide is not less than 4000.

10. A radiation-resistant medical molded article fabricated using a resin composition containing the radiation-resistant medical polyamide resin composition according to claim 1.

11. The radiation-resistant medical molded article according to claim 10, which is a medical tube or a medical balloon.

12. A radiation-resistant resin additive that contains, as effective ingredient, a bisphenol compound as indicated by General Formula (1) below, or General Formula (2) below; 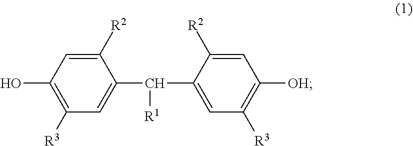
wherein R¹, R², and R³ respectively may be the same or different and indicate a hydrogen atom or a saturated hydrocarbon group having not less than 1 carbon; 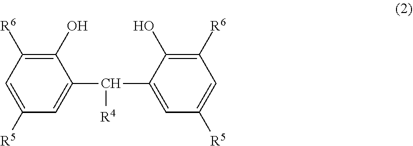
wherein R⁴, R⁵, and R⁶ respectively may be the same or different and indicate a hydrogen atom or a saturated hydrocarbon group having not less than 1 carbon.
